(19) United States
(12) Patent Application Publication
(10) Pub. No.: US 2006/0231443 A1
(43) Pub. Date: Oct. 19, 2006

(54) Method of packaging and sterilizing disposable articles for surgical operations and such a package

(76) Inventors: Peter Jonasson, Lerum (SE); Simon Smith, Hill Farm (GB); Jean-Michel Van Der Hofstadt, Hannut (BE); Phil Cooper, North Marston (GB)

Correspondence Address: YOUNG & THOMPSON, 745 SOUTH 23RD STREET, 2ND FLOOR, ARLINGTON, VA, 22202, US

(21) Appl. No.: 10/543,400

(22) PCT Filed: Feb. 13, 2004
(86) PCT No.: PCT/SE04/00189

§ 371 Date: Jan. 19, 2006

(30) Foreign Application Priority Data
Feb. 25, 2003 (SE) 0300496-7

(51) Int. Cl. A61B 19/02; B65B 55/02; A61L 2/18; A61L 2/00; B65D 83/10

(52) U.S. Cl. 206/439; 53/425; 206/370; 422/28; 422/300

(57) ABSTRACT

A method of packaging and sterilizing disposable articles for surgical operations, in which several surgical articles are packed in a first package that is packed in a transport package and thereafter transported to a plant for sterilizing with the aid of ethylene oxide, whereafter the first package is sterilized in this plant. A second package containing sterile packaged surgical articles of a type, that can not sustain sterilizing, or for other reasons should not be sterilized with ethylene oxide, is brought together with the first package before this package is packed into the transport package and before this package is transported to the sterilizing plant, the second package being impermeable to ethylene oxide. A transport package used in the method and a package for sterile packaged articles, which can not sustain sterilizing or should not be sterilized with ethylene oxide is also disclosed.

### TECHNICAL FIELD

The present invention relates to a method of packaging and sterilizing disposable articles for surgical operations or interventions, in which several surgical articles needed for a certain surgical operation, are packed in a first package that is packed in a transport package and thereafter transported to a plant for sterilization with the aid of ethylene oxide, whereafter the first package is sterilized in this plant and to a package provided by the method.

### BACKGROUND OF THE INVENTION

As a part of taking a greater total responsibility for the handling of sterile disposable articles for surgical operations or interventions it has became common to deliver all sterile articles in a so called tray. This tray contains all or most of the disposable articles for a specific type of operation, such as surgical coats, drapes for patients and instruments, incisions films, tubes, scalpels, drainage, bowls, sutures, etc. Such a tray can be plastic bag, a paper bag or a bag of nonwoven material or the like. An advantage for the customer is that the trays save time in gathering all equipment before surgical operation or intervention. This time can instead be spent on the well-being of the patient. Furthermore, the refuse handling of the hospital is greatly reduced when the amount of single-packed articles is reduced. The storing of different articles in the hospital becomes reduced and the inventory will be easier to perform. The cost for each operation is likely to be reduced for the hospital as a consequence.

Surgical drapes, surgical coats, etc are not handled in a sterile manner when put in a tray. After sealing of the package, it is put in a transport package together with one or more other trays and is transported to a sterilization plant that can be located a long way from the packaging plant, even in another country. In the sterilization plant the packaged as a whole is sterilized with the aid of ethylene oxide (EtO). By this technique of sterilization the package can be subjected to sequences of atmospheres of steam and EtO and also cycles of pressure variations of both high and low pressure. This sterilization method requires costly packages that are permeable to EtO but not to other substances. Moreover, EtO is both explosive and toxic. Some articles are sensitive to EtO-gas or sensitive to high or low pressure. Such articles must be handled in a special way. Another reason for the need of handling articles in a special way is that the deliverer will not guarantee the quality of the product after such handling, and will consequently not take responsibility for possible faults that can appear. An antimicrobic incision film Ioban from 3M, St Paul, Minn. is an example of an article, which must not be subjected to sub-pressure. The active substance in this material is “iodophor” which is stable at normal pressure but evaporates at sub-pressure. When iodophor vanishes from the material, the incitement to use Ioban has also disappeared. Another example of products which should not be subjected to EtO under large sub-pressure is a suture, Suture Mersilk black Sz.4 from Ethicon, Ireland.

In order to be able to deliver Ioban, and other materials that can not sustain EtO-sterilization, in a tray it is common to use a so-called piggyback. This piggyback consists of a bag, which is taped on to a tray after the EtO-sterilization has been performed. A piggyback contains, in single sterile packages, the articles, which must not be EtO-sterilized and which together with the articles in a tray are needed for a certain type of surgical operation. These back-packs are transported separate from the transport packages containing the trays to the sterilization plant and are there brought together with the associated tray after this has been sterilized.

A problem with the today's handling of piggybacks is that after the EtO-sterilization one has to open the sterilized transport packages, which can contain two or more trays, in order to tape a piggyback on to a tray. This is not only a problem of logistics but can also lead to quality problems by missing to attach a piggyback or to attach the wrong piggyback. A faulty combination of tray and piggyback delivered to a hospital leads to problems at preparation for a surgical operation and to an increase of refuse since both tray and piggyback in the faulty combination are disposed of. The handling of today increases the total cost for a tray, since the transport packages after sterilization have to be opened and resealed.

The objective of the present invention is to solve these problems and simplify the handling of trays and piggybacks.

### SUMMARY OF THE INVENTION

These objectives are according to the invention accomplished by a method of packaging and sterilizing disposable articles for surgical operations, in which several surgical articles needed for a certain surgical operation, are packed in a first package that is packed in a transport package and thereafter transported to a plant for sterilizing with the aid of ethylene oxide, whereafter the first package is sterilized in this plant, characterised in that a second package containing sterile packaged surgical articles of a type, that can not sustain sterilizing with the aid of ethylene oxide or for other reasons should not be sterilized with the aid of ethylene oxide, is brought together with the first package before this package is packed into the transport package and before this package is transported to the sterilizing plant, whereby the second package is impermeable to ethylene oxide. By using a piggyback bag impermeable to EtO, tray and correct piggyback can already before sterilization be brought together. Since gases, such as steam and EtO, cannot penetrate into the product inside the piggyback bag will exposure to, evaporation, chemical reactions, etc with EtO not take place. By using a piggyback impermeable to EtO and place it in the transport package before sterilization one is spared the handling of piggyback separately after sterilization and all the disposable articles needed for a certain type of surgical operation can be packed at the same time and at the same location. Consequently, several quality and logistic problems are then solved and at the same time trays can be manufactured in a more economic manner.

In a variant, the second package can be placed within the first package.

The invention also relates to a transport package containing a first package of surgical articles needed for a certain type of surgical operation, in which first package the articles are sterilized with the aid of ethylene oxide characterised in that the transport-package comprises a second package, in which sterile packed articles, which can not sustain sterilizing with the aid of ethylene oxide or for other reasons should not be sterilized with the aid of ethylene oxide, are disposed, the articles in the second package being needed for the same type of surgical operation as the articles in the first package and the second package is impermeable to ethylene oxide.

In a preferred embodiment, the second package is made of a laminate of one or more plastic layers and aluminium foil and the first package is made of air permeable plastic film, e.g. polyethylene, a so called breatherbag, or a bag of nonwoven material.

The invention further relates to a package for sterile packaged articles, which can not sustain sterilizing with the aid of ethylene oxide or for other reasons should not be sterilized with the aid of ethylene oxide, characterised in that it is made of a laminate of one or more plastic layers and aluminium foil.

### BRIEF DESCRIPTION OF THE DRAWING

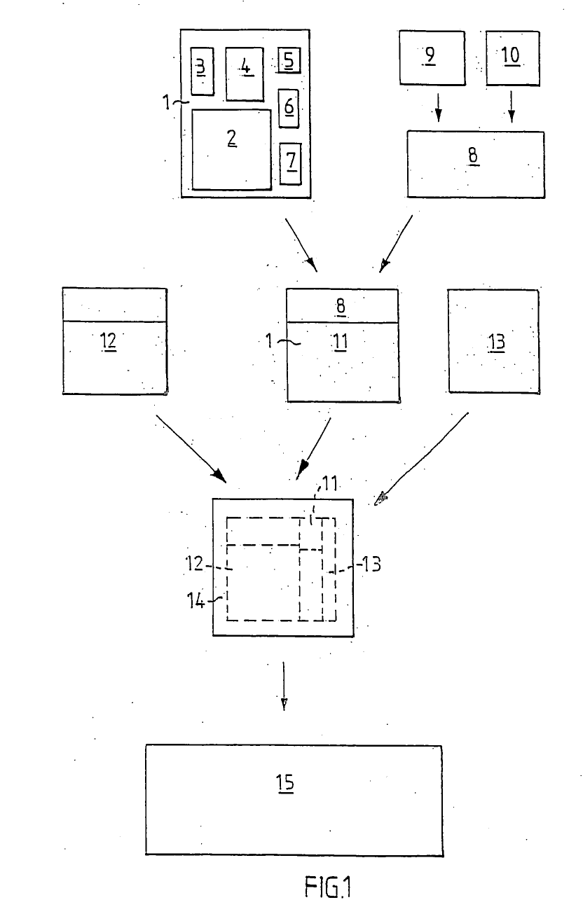

The invention will now be described with reference to FIG. 1 schematically illustrating the different steps in a method according to the invention.

### DESCRIPTION OF EMBODIMENTS

In a first package 1, a so-called tray, are packed the unsterilized disposable articles 2-7 needed for a surgical operation of a certain type. Such articles typically consist of surgical coats, patient and instrument drapes for the surgical operation in question, tubes, bandages, wound dressings and instruments, such as bowls, drainage, etc. The first package 1 is thereafter sealed. The first package can be a bag made of a material, which is permeable to gas of ethylene oxide but impermeable to microorganisms.

In a second package 8, a so called piggyback, are packed sterile packed disposable articles 9,10 which are needed for the same type of surgical operation as articles 2-7 in the first package. Articles 9,10 are such that they for one or other reasons must not be sterilized by gas of ethylene oxide and they are delivered in sterile packages to the packaging location. An example of such an article is an incision film “Ioban” from 3M, St Paul, Minn., USA. This article contains an active substance “iodophor” which is stable at normal pressure but evaporates at sub-pressure. By the fact that the sterilizing process means that the first package will be subjected to a cycle of overpressure and/or sub-pressure, such an incision film can not be packed in the first package without causing its active substance to vanish from the film. “Ioban” is delivered from the manufacturer in a sterile package. When the articles 9,10 have been packed in the second package 8, the package 8 is sealed.

The second package 8 can consist of a bag made of a laminate of one or more plastic layers and a film of aluminium foil. An example of such a material is PerfecFlex 35781-G from Perfecseal, Londondeny, Northern Ireland, which is impermeable to gas of EtO. A bag in this material is suitably sealed at one or more sides with the aid of impulse welding. The other sides can, as a suggestion, be sealed by ultra sonic welding or by adhesive.

The first and second packages 1 and 8 are thereafter brought together and the second package 8 is attached to the first package with the help of a tape or the like. All the disposable articles 2-7,9,10 needed for the surgical operation in question are thereby gathered to a single unit 11.

It is of course possible to dispose the second package 8 within the first package 1 before this package is sealed.

The unit 11 is then placed in a transport package 14 alone or together with other units 12,13, which can contain articles for the same type of surgical operation or other types of surgical operations. The transport package 14 is manufactured of a material permeable to gas of ethylene oxide or made permeable to gas of ethylene oxide, e.g. paper, cardboard or the like.

The transport package 14 is then brought to a sterilization plant 15, in which the articles in the first packages are sterilized by being subjected to an atmosphere of ethylene gas under a varying pressure cycle, in which the pressure is alternately varied from a very low sub-pressure (80 mbar) to atmospheric pressure (1013 mbar). In an alternative sequence of sterilization the pressure can be alternated between atmospheric pressure (1013 mbar) and overpressure (4000 mbar). Due to the impermeability to gas of ethylene oxide of the second packages 8, located in the transport packages 14, the content of these packages will not be influenced by this sterilizing step. Other pressure cycles and pressures can also be used and steam can also be supplied during the sterilizing process. After sterilization, the transport packages are sent to customers.

The sterilization plant 15 can be situated at a location different from the packaging location and can even be situated in another country. By the fact that the first and second packages can be brought together and packed in the transport package before it is sent to the sterilization plant all disposable articles needed for a certain surgical operation can be packed and brought together at the same location. Advantages relative to logistic and reduced risk for the second packages to be matched with the wrong type of first packages are thereby obtained.

The described embodiment shall only be taken as an example to illustrate the principle behind the invention. More articles than shown can thus be packed into the first package depending on the type of surgical operation the package is intended for. The same is of course valid for the second package and the transport package of units. Furthermore, the units consisting of tray and piggyback can be packed in a further protection package, e.g. an open plastic bag or the like, in order to allow the units to be taken out of the transport packages for intermediate storing in the hospital without risk for breaking the sterility.

1-6. (canceled)

7. Method of packaging and sterilizing disposable articles (2-7) for surgical operations, in which several surgical articles needed for a certain surgical operation are packed in a first package (1) that is packed in a transport package (14) and thereafter transported to a plant (15) for sterilizing with the aid of ethylene oxide, whereafter the first package is sterilized in this plant, characterized in that a second package (8) containing sterile packaged surgical articles (9,10) of a type, that can not sustain sterilizing with the aid of ethylene oxide or for other reasons should not be sterilized with the aid of ethylene oxide, is brought together with the first package (1) before this package is packed into the transport package and before this package is transported to the sterilizing plant, whereby the second package is impermeable to ethylene oxide.

8. Method according to claim 7, characterized in that the second package (8) is placed within the first package (1).

9. Transport package (14) containing a first package (1) of surgical articles (2-7) needed for a certain type of surgical operation, in which first package the articles are sterilized with the aid of ethylene oxide, characterised in that the transport package comprises a second package (8), in which sterile packaged articles (9,10), which can not sustain sterilizing with the aid of ethylene oxide or for other reasons should not be sterilized with the aid of ethylene oxide, are disposed, the articles in the second package being needed for the same type of surgical operation as the articles in the first package and the second package is impermeable to ethylene oxide.

10. Package according to claim 9, characterised in that the second package (8) is made of a laminate of one or more plastic layers and aluminium foil.

11. Package according to claim 9, characterised in that the first package (1) is made of air permeable plastic film, paper or nonwoven.

12. Package according to claim 10, characterised in that the first package (1) is made of air permeable plastic film, paper or nonwoven.
